(19) United States
(12) Patent Application Publication
(10) Pub. No.: US 2023/0330114 A1
(43) Pub. Date: Oct. 19, 2023

(54) COMPOSITIONS COMPRISING MONO- AND DI-GLYCERIDES WITH OMEGA-3 FATTY ACYL GROUPS AND METHODS OF USING SAME

(71) Applicant: ENZYMOCORE LTD., Shfar-Am (IL)

(72) Inventors: Sobhi BASHEER, Sakhnine (IL); Ramez MASRI, Nahef Village (IL)

(21) Appl. No.: 18/136,596

(22) Filed: Apr. 19, 2023

Related U.S. Application Data
(63) Continuation of application No. PCT/IL2021/051263, filed on Oct. 25, 2021.
(60) Provisional application No. 63/105,421, filed on Oct. 26, 2020.

(51) Int. Cl. A61K 31/00; A61K 31/202

(52) U.S. Cl. CPC A61K 31/658; A61K 31/202

(57) ABSTRACT

Compositions containing mono- and di-glycerides with at least 20% (w/w) omega-3 fatty acyl groups and methods of utilizing same.

### CROSS REFERENCE TO RELATED APPLICATIONS

This application is a Bypass Continuation of PCT Patent Application No. PCT/IL2021/051263 having International filing date of Oct. 25, 2021, which claims the benefit of priority of U.S. Provisional Pat. Application No. 63/105,421, filed Oct. 26, 2020, the contents of which are all incorporated herein by reference in their entirety.

### FIELD OF INVENTION

The present invention is directed generally to compositions containing mono- and di-glycerides with omega-3 fatty acyl groups and method for using same for elevating the levels of endocannabinoids in a subject in need thereof and for treatment of inflammation in particular pancreatic and/or liver inflammation. The invention also relates to compositions containing mono-and di-glycerides with omega-3 fatty acyl groups and a phytocannabinoid and/or Vitamin D as well as method of utilizing same for increasing the bioavailability of the phytocannabinoids.

### BACKGROUND OF THE INVENTION

Polyunsaturated fatty acids (PUFA) represent a group of long-chain fatty acids containing two or more double bonds. PUFA are subdivided into two main groups; omega-3 fatty acids and omega-6 fatty acids. The omega-3 fatty acids group includes the essential fatty acids α-linolenic acid (ALA, 18:3, omega-3) and its long-chain metabolites, namely, EPA (eicosapentaenoic acid), DHA (docosahexaenoic acid) and DPA (docosapentaenoic acid), while the omega-6 fatty acids group includes arachidonic acid (ARA) and linoleic acid [Gill, I. and Valiverty, R., Part 1: Trends in Biotechnology, 1997, 15:401. Part 2: Trends in Biotechnology. 1997, 15:470].

Omega-3 and omega-6 are classified as “essential fatty acids” because they cannot be produced by the human body and therefore EPA and DHA in particularly must be obtained through other sources, such as fish and oleaginous oils [Inform, Meeting probes n-3 fatty acids’ medical role. 1997, 8(2):176; Inform, Nutritional aspects of n-3 fatty acids. 1997, 8(5):428].

Many epidemiological studies suggest that the omega-3 fatty acids DHA and EPA promote beneficial cardiovascular, neurological and inflammatory health effects. It has also been suggested that the omega-3 fatty acids EPA and DHA undergo biochemical conversion to yield eicosapentaenoyl ethanolamide (EPEA) and docosahexaenoyl ethanolamide (DHEA), respectively, which are known as omega-3 endocannabinoids. Similar to arachidonoyl ethanolamine (AEA) known as anandamide both components EPEA and DHEA can activate cannabinoid receptor-1 (CB1) which is found predominantly in the central nerve system (CNS), and receptor-2 (CB2) which is found in both peripheral and CNS immune cells. EPAE has been shown to activate anti-inflammatory pathways while DHEA was found to exhibit anticancer, anti-inflammatory, and synaptogenic properties.

Omega-3 fatty acids in the form of free fatty acids or lower alkyl esters of methyl or ethyl alcohols are commercially available in high purity. These omega-3 fatty acids derivatives are usually used for the preparation of triglycerides with high omega-3 content. However, omega-3 triglycerides are primarily used for energy consumption and storage in the form of esters, phospholipids, ethers, glycolipids, sphingolipids and lipoproteins.

Numerous methods have been applied separately or in combination to concentrate, separate and recover omega-3 fatty acids and their derivatives (methyl or ethyl esters, triglycerides and amides) from naturally occurring sources, such as fish and oleaginous oils. These methods include mainly fractional crystallization at low temperatures, molecular distillation, urea adduct crystallization, stationary bed chromatography and with lipases. Lipases are defined as hydrolytic enzymes that act on the ester linkages in triacylglycerol molecules in an aqueous system to yield free fatty acids, partial glycerides and glycerol and have been shown by the inventors of the present invention to offer an attractive alternative method compared to conventional physico-chemical processes for the enrichment and concentration of omega-3 fatty acids in different forms.

Of particular interest, lipases of selective transesterification/esterification activity have been used for the preparation of omega-3 fatty acids in the form of glycerides (mono-, di- and tri-glycerides) with different ratios. In this type of reactions, omega-3 fatty acids-containing oil, such as fish or oleaginous oils, is reacted with a short-chain alcohol, preferably ethanol, in the presence of lipase with low selectivity towards omega-3 fatty acyl groups and high tolerance to ethanol. Lipase can be used in its free or immobilized forms. The reaction medium after treatment with such a lipase is filtered off, excess of alcohol and water are removed after phase separation of the reaction mixture and then are removed from the upper oil phase by flash evaporation. The formed fatty acids short-chain alkyl esters, preferably ethyl esters, predominantly with low content of omega-3 fatty acyl groups are distilled off under high vacuum distillation (such as short-path, thin-film or molecular distillation) from the residual oil phase to obtain a mixture of glycerides (mono-, di- and tri-glycerides) containing higher than 50% omega-fatty acids. The reaction mixture after distillation optionally contains free fatty acids and fatty acids short-chain alkyl esters less than 20% by weight. Depending on the type of the starting material and lipase selectivity used in the transesterification/esterification reaction a predetermined ratio of omega-3 fatty acids in the form of glycerides can be obtained.

### SUMMARY OF THE INVENTION

The present invention relates to compositions containing mono- and di-glycerides with omega-3 fatty acyl groups and method for using same for elevating the levels of endocannabinoids in a subject in need thereof. As demonstrated herein below it was found that administering compositions comprising mono- and di-glycerides with omega-3 fatty acyl groups to mice caused a significant increase in the level of various endocannabinoids in both the plasma and the brain, advantageously without triggering pain as opposed to arachidonic acid triggered endocannabinoid production.

The herein disclosed compositions may advantageously be used for treatment of inflammation in particular pancreatic and liver inflammation as well as other diseases such as CNS disorders, such as epilepsy, Parkinson, rheumatic disorders, diabetes, ADD and ADHD. As further demonstrated herein below, administration of the herein disclosed compositions to mice advantageously caused a significant decrease in blood serum hydrolases, such as lipase and/or amylase, thus indicating a reduced degree of inflammation.

Advantageously, it was further found that compositions containing a phytocannabinoid in addition to the mono- and di-glycerides with omega-3 fatty acyl groups significantly increase the bioavailability of the phytocannabinoid.

According to some embodiments, the composition induces a synergistic effect between the mono- and di-glycerides containing omega-3 fatty acyl groups, the phytocannabinoid. Each possibility is a separate embodiment.

According to some embodiments, there is provided a method for increasing the levels of omega-3 fatty acid-based endocannabinoids in a subject in need thereof, the method comprising administering to the subject a composition comprising mono- and di-glycerides containing at least 20% (w/w) of the total product weight omega-3 fatty acyl groups, thereby increasing the level of thereby increasing the level of the omega-3 fatty acids and the omega-3 fatty acid-based endocannabinoids.

According to some embodiments, the mono- and di-glycerides contain at least 50% (w/w) or at least 60% (w/w) omega-3 fatty acyl groups omega-3 fatty acyl groups.

According to some embodiments, the omega-3 fatty acyl groups include eicosapentaenoic acid (EPA) fatty acyl groups, docosahexaenoic acid (DHA) fatty acyl groups or both. Each possibility is a separate embodiment.

According to some embodiments, the composition comprises mono- and di- glycerides containing one or more fatty acyl groups selected from oleic, palmitic, myristic, stearic, arachidonic, palmitoleic, linoleic and linolenic acyl groups. Each possibility is a separate embodiment.

According to some embodiments, administration of the composition induces an increase of the levels of 2-monoglyceride and/or fatty acid ethanol amides of omega-3 fatty acyl groups, such as but not limited to EPA and DHA, in the subject.

According to some embodiments, administration of the composition induces an increase of one or more biological effects that are mediated primarily by CB1 cannabinoid receptors in the central nervous system, and/or CB2 cannabinoid receptors in the periphery.

According to some embodiments, administration of the composition reduces or prevents one or more undesired biologic effect caused by arachidonic acid-based endocannabinoids.

According to some embodiments, the composition further comprises tri-glycerides containing at least one omega-3 fatty acyl group. According to some embodiments, the tri-glycerides comprise at least one omega-3 fatty acyl group.

According to some embodiments, the composition comprises less than 20% w/w free fatty acids.

According to some embodiments, the composition comprises less than 20% w/w fatty acid ethyl esters.

According to some embodiments, the composition comprises at least 10% w/w omega-3 mono-glycerides.

According to some embodiments, the composition comprises at least 10% w/w omega-3 di-glycerides.

According to some embodiments, the ratio of mono-glycerides and di-glycerides in the composition is in the range of about 1.5:1 - 1:6.

According to some embodiments, there is provided a method for treating inflammation in a subject in need thereof, the method comprising administering to the subject a composition comprising mono- and di-glycerides containing at least 20% (w/w) omega-3 fatty acyl groups, thereby reducing the level of inflammation in the subject.

According to some embodiments, reducing the level of inflammation comprises reducing the level and/or activity of blood serum hydrolases. According to some embodiments, the blood serum hydrolases comprise lipase and/or amylase. According to some embodiments, reducing the level of inflammation comprises reducing the level and/or activity of one or more aminotransferases. According to some embodiments, the one or more aminotransferases comprises alanine aminotransferase (ALT) and/or aspartate aminotransferase (AST). According to some embodiments, reducing the level of inflammation comprises reducing the level of one or more cytokines. According to some embodiments, the one or more cytokines is selected from IL1-β, TNF-α, IL-4, MCP-1, IL-6, IL-10 or any combination thereof. Each possibility is separate embodiment.

According to some embodiments, the inflammation is pancreatic inflammation. According to some embodiments, the inflammation is liver inflammation.

According to some embodiments, there is provided a composition comprising mono- and di-glycerides containing at least 20% (w/w) omega-3 fatty acyl groups, wherein the composition comprises at least 10% (w/w) mono-glycerides containing at least 20% (w/w) omega-3 fatty acyl groups; or at least 10% (w/w) di-glycerides containing at least 20% (w/w) omega-3 fatty acyl groups; or wherein the ratio of mono-glycerides and di-glycerides in the composition is in the range of about 1.5:1 - 1:6. Each possibility is a separate embodiment.

According to some embodiments, the mono- and/or di-glycerides comprises at least 50% (ww) omega-3 fatty acyl groups.

According to some embodiments, the composition further comprises tri-glycerides containing at least 20% (w/w) and preferably at least 50% (w/w) omega-3 fatty acyl groups.

According to some embodiments, the composition comprises less than 20% (w/w) free fatty acids.

According to some embodiments, the composition comprises less than 20% (w/w) fatty acids ethyl esters.

According to some embodiments, the composition is suitable for use in increasing omega-3 fatty acid- based endocannabinoids in a subject.

According to some embodiments, the composition is suitable for use in increasing mono-, di- and/or tri-unsaturated fatty acid-based endocannabinoids in a subject.

According to some embodiments, the composition is suitable for use in treating inflammation. According to some embodiments, the inflammation is pancreatic inflammation. According to some embodiments, the inflammation is liver inflammation.

According to some embodiments, the composition comprises at least 10% (w/w) di-glycerides.

According to some embodiments, the ratio of mono-glycerides and di-glycerides in the composition is in the range of about 1.5:1 - 1:6.

According to some embodiments, the composition comprises less than 20% w/w free fatty acids.

According to some embodiments, the composition is suitable for use in increasing omega-3 fatty acid-based endocannabinoids.

According to some embodiments, the composition is suitable for use in increasing mono-, di- and tri-unsaturated fatty acid-based endocannabinoids.

According to some embodiments, the composition is suitable for use in treating inflammation. According to some embodiments, the inflammation is pancreatic inflammation. According to some embodiments, the inflammation is liver inflammation.

According to some embodiments, the composition further comprises one or more phytocannabinoid. According to some embodiments, the phytocannabinoid comprises cannabidiol (CBD) and/or tetrahydrocannabinol (THC). According to some embodiments, the composition comprises at least about 0.01% (w/w) phytocannabinoid. According to some embodiments, the composition increases the bioavailability of the phytocannabinoid.

According to some embodiments, the composition is formulated for oral, intraperitoneal (IP), intravenous (IV), or subcutaneous (SC) administration. Each possibility is a separate embodiment.

According to some embodiments, the composition in the form of emulsion, suspension, liposomes, encapsulated in capsules or confined in hydrogels. Each possibility is a separate embodiment.

According to some embodiments, there is provided a method for increasing bioavailability of a phytocannabinoid, the method comprising administering to a subject in need thereof the herein disclosed phytocannabinoid composition.

According to some embodiments, the composition further comprises vitamin D. According to some embodiments, the composition comprises at least about 0.01% (w/w) Vitamin D. According to some embodiments, the composition increases the bioavailability of the Vitamin D.

According to some embodiments, the composition is formulated for oral, intraperitoneal (IP), intravenous (IV), or subcutaneous (SC) administration. Each possibility is a separate embodiment.

According to some embodiments, the composition is in the form of emulsion, suspension, liposomes, encapsulated in capsules or confined in hydrogels. Each possibility is a separate embodiment.

According to some embodiments, there is provided a method for increasing bioavailability a phytocannabinoid, the method comprising administering to a subject in need thereof the herein disclosed composition including a phytocannabinoid.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

The invention will now be described in relation to certain examples and embodiments with reference to the following illustrative figures so that it may be more fully understood.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures so that it may be more fully understood.

FIG. 1A shows a presentation of the Mean plasma concentrations of EPA in ppm in plasma serum. The EPA values were evaluated 0, 0.5, 1.5, 4, 8 and 24 hours post administration. Results are presented as mean ± SEM.

FIG. 1B shows a presentation of the Mean plasma concentrations of DHA in ppm in plasma serum. The DHA values were evaluated 0, 0.5, 1.5, 4, 8 and 24 hours post administration. Results are presented as mean ± SEM.

FIG. 2 shows representative liver microscopy images obtained from mice untreated or treated with carbon tetrachloride (CC14) (upper and lower panels, respectively) and administered with soybean oil (SBO - left panels) or with the herein disclosed composition (ENZYMEGA -right panels).

FIG. 3 shows the average liver wight of mice untreated or treated with CCl4 (white and grey columns, respectively) and administered with soybean oil (SBO) or with the herein disclosed composition (ENZYMEGA).

FIG. 4 shows representative H&E staining of liver tissues obtained from mice untreated or treated with CCl4 (upper and lower panels, respectively) and administered with soybean oil (SBO - left panels) or with the herein disclosed composition (ENZYMEGA - right panels).

FIG. 5 shows representative Sirius Red staining of liver tissues obtained from mice untreated or treated with CCl4 (upper and lower panels, respectively) and administered with soybean oil (SBO - left panels) or with the herein disclosed composition (ENZYMEGA - right panels).

FIG. 6A shows average ALT levels in the blood of mice untreated or treated with CCl4 (white and grey columns, respectively) and administered with soybean oil (SBO) or with the herein disclosed composition (ENZYMEGA).

FIG. 6B shows average ALT levels in the blood of mice untreated or treated with CCl4 (white and grey columns, respectively) and administered with soybean oil (SBO) or with the herein disclosed composition (ENZYMEGA).

FIG. 7A shows average IL-1β levels in the blood of mice untreated or treated with CCl4 (white and grey columns, respectively) and administered with soybean oil (SBO) or with the herein disclosed composition (ENZYMEGA).

FIG. 7B shows average TNF-α levels in the blood of mice untreated or treated with CCl4 (white and grey columns, respectively) and administered with soybean oil (SBO) or with the herein disclosed composition (ENZYMEGA).

FIG. 7C shows average IL-4 levels in the blood of mice untreated or treated with CCl4 (white and grey columns, respectively) and administered with soybean oil (SBO) or with the herein disclosed composition (ENZYMEGA).

FIG. 7D shows average MCP-1 levels in the blood of mice untreated or treated with CCl4 (white and grey columns, respectively) and administered with soybean oil (SBO) or with the herein disclosed composition (ENZYMEGA).

FIG. 7E shows average IL-6 levels in the blood of mice untreated or treated with CCl4 (white and grey columns, respectively) and administered with soybean oil (SBO) or with the herein disclosed composition (ENZYMEGA).

FIG. 7F shows average IL-10 levels in the blood of mice untreated or treated with CCl4 (white and grey columns, respectively) and administered with soybean oil (SBO) or with the herein disclosed composition (ENZYMEGA).

FIG. 7G shows average IL-2 levels in the blood of mice untreated or treated with CCl4 (white and grey columns, respectively) and administered with soybean oil (SBO) or with the herein disclosed composition (ENZYMEGA).

FIG. 7H shows average IFN-γ levels in the blood of mice untreated or treated with CCl4 (white and grey columns, respectively) and administered with soybean oil (SBO) or with the herein disclosed composition (ENZYMEGA).

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The following definitions and methods are provided to better define the present invention and to guide those of ordinary skill in the art in the practice of the present invention. Unless otherwise noted, terms are to be understood according to conventional usage by those of ordinary skill in the relevant art.

Unless otherwise defined, all terms of art, notations and other scientific terminology used herein are intended to have the meanings commonly understood by those of ordinary skill in the art to which the present application pertains. In some cases, terms with commonly understood meanings are defined herein for clarity and/or for ready reference, and the inclusion of such definitions herein should not necessarily be construed to represent a substantial difference over what is generally understood in the art.

Where a term is provided in the singular, the inventors also contemplate aspects of the invention described by the plural of that term.

It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

As used herein, the terms “approximately” and “about” refer to +/-10%, or +/-5%, or +-2% vis-à-vis the range to which it refers. Each possibility is a separate embodiment.

As used herein, the terms “glyceride” and “acylglycerol” may be used interchangeably and refer to esters formed from glycerol and fatty acids. Glycerol has three hydroxyl functional groups, which can be esterified with one, two, or three fatty acids to form mono-, di-, and triglycerides.[2] These structures vary in their fatty acid carbon chains as they can contain different carbon numbers, different degrees of unsaturation, and different configurations and positions of olefins.

As used herein the term “fatty acid” refer to a carboxylic acid with a long aliphatic chain, which is either saturated or unsaturated. Fatty acids are usually not found in organisms in their standalone form also referred to as “free fatty acids”, but rather as one of three main classes of esters: triglycerides, phospholipids, glycolipids, and cholesteryl esters. As used herein the terms “fatty acid ethyl esters” and “FAEE” refer to a type of ester that result from the combination of a fatty acid with an alcohol.

As used herein, the terms “Eicosapentaenoic acid”, “EPA”, “icosapentaenoic acid” and “timnodonic acid” may be used interchangeably and refer to an omega-3 fatty acid. In physiological literature, it is given the name 20:5(n-3). In chemical structure, EPA is a carboxylic acid with a 20-carbon chain and five cis double bonds; the first double bond is located at the third carbon from the omega end. EPA is a polyunsaturated fatty acid (PUFA) that acts as a precursor for prostaglandin-3, thromboxane-3, and leukotriene-5 eicosanoids. EPA is both a precursor and the hydrolytic breakdown product of eicosapentaenoyl ethanolamide (EPEA: C22H35NO2; 20:5, n-3).

As used herein, the terms “Docosahexaenoic acid”, “DHA” and “cervonic acid” may be used interchangeably and refer to an omega-3 fatty acid that is a primary structural component of the human brain, cerebral cortex, skin, and retina. In physiological literature, it is given the name 22:6(n-3). It can be synthesized from alpha-linolenic acid or obtained directly from breast milk, fish oil, or algae oil. DHA’s structure is a carboxylic acid (-oic acid) with a 22-carbon chain and six cis double bonds with the first double bond located at the third carbon from the omega end.

As used herein, the terms “cannabinoid” or “cannabinoid compound” and “phytocannabinoid” may be interchangeable and may refer to a member of a class of unique meroterpenoids synthesized by plants. Cannabinoids activate cannabinoid receptors (CB1 and CB2).

Non-limiting examples of cannabinoids include: cannabichromene (CBC) type (e.g. cannabichromenic acid), cannabigerol (CBG) type (e.g. cannabigerolic acid), cannabidiol (CBD) type (e.g. cannabidiolic acid), Δ9-trans-tetrahydrocannabinol (Δ9 -THC) type (e.g. Δ9-tetrahydrocannabinolic acid), Δ8-trans-tetrahydrocannabinol (Δ8 -THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), Δ9-tetrahydrocannabinolic acid A (THCA-A), Δ9-tetrahydrocannabinolic acid B (THCA-B), Δ9-tetrahydrocannabinol (THC), Δ9-tetrahydrocannabinolic acid-C4 (THCA-C4), Δ9-tetrahydrocannabinol-C4 (THC-C4), Δ9 -tetrahydrocannabivarinic acid (THCVA), Δ9-tetrahydrocannabivarin (THCV), Δ9- tetrahydrocannabiorcolic acid (THCA-C1), Δ9-tetrahydrocannabiorcol (THC-C1), Δ7-cis-iso-tetrahydrocannabivarin, Δ8-tetrahydrocannabinolic acid (Δ8-THCA), Δ8- tetrahydrocannabinol (Δ8-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN-C4), cannabivarin (CBV), cannabinol-C2 (CNB-C2), cannabiorcol (CBN-C1), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

As used herein, the term “Endocannabinoid” refers endogenous ligands of cannabinoid receptors (CB1 and CB2).

As used herein, the term “bioavailability” refers to the rate and extent to which a drug reaches a site of action.

The term “treating” as used herein refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of spread or development of the disease or condition, delay or slowing of disease progression, amelioration or palliation of the disease state, and remission (whether partial or total). “Treating” can also mean prolonging survival of a patient beyond that expected in the absence of treatment. “Treating” can also mean inhibiting the progression of disease, slowing the progression of disease temporarily, although more preferably, it involves halting the progression of the disease permanently.

As used herein, the term “subject” may refer to any mammal. According to some embodiments, the subject is a human.

According to some embodiments, there is provided a composition containing mono- and di-glycerides also referred to herein as “acylglycerols” containing at least 20% (w/w) of the total fatty acids weight omega-3 fatty acyl groups.

According to some embodiments, the composition contains at least 5%, at least 10%, at least 20%, or at least 30% (w/w) of the mono-glycerides with at least 20% (w/w) omega-3 fatty acyl groups. Each possibility is a separate embodiment.

According to some embodiments, the composition contains at least 5%, at least 10%, at least 20%, or at least 30% (w/w) of the di-glycerides containing at least 20% (w/w) omega-3 fatty acyl groups.

According to some embodiments, the ratio of the mono-glycerides and the di-glycerides in the composition is in the range of about 5:1 - 1:15. According to some embodiments, the ratio of the mono-glycerides and the di-glycerides in the composition is in the range of about 3:1 - 1:10. According to some embodiments, the ratio of the mono-glycerides and the di-glycerides in the composition is in the range of about 1.5:1 - 1:6.

According to some embodiments, the mono- and di-glycerides contain at least 30%, at least 40%, at least 50%, or at least 60% (w/w) omega-3 fatty acyl groups. Each possibility is a separate embodiment.

According to some embodiments, the omega-3 fatty acyl groups are eicosapentaenoic acid (EPA) fatty acyl groups, docosahexaenoic acid (DHA) fatty acyl groups or both. Each possibility is a separate embodiment.

According to some embodiments, the composition further contains mono- and di-glycerides with one or more fatty acyl groups selected from oleoyl, palmitoleoyl, linoleoyl and linoleneoyl, and arachidonoyl acyl groups. Each possibility is a separate embodiment.

Without being bound by any theory, administration of the composition induces an increase in the levels of 2-monoglyceride and/or fatty acid ethanol amides of omega-3 fatty acyl groups, such as EPA and DHA, and optionally also of non-omega-3 fatty acyl groups in the subject.

Without being bound by any theory, administration of the composition induces an increase of one or more biological effects that are mediated primarily by CB1 cannabinoid receptors in the central nervous system, and/or CB2 cannabinoid receptors in the periphery.

Without being bound by any theory, administration of the composition reduces or prevents one or more undesired biologic effect, such as pain and thrombosis caused by arachidonic acid-based endocannabinoids and/or arachidonic acid pathways. According to some embodiments, administration of the composition inhibits formation of mono- and di-acyl glycerol lipases, typically involved in increasing the levels of inflammatory endocannabinoids, such as arachidonoyl glycerol arachidonoyl ethanol amide in body tissues.

According to some embodiments, the composition further contains tri-glycerides having at least one omega-3 fatty acyl group. According to some embodiments, the tri-glycerides comprise at least one omega-3 fatty acyl group.

According to some embodiments, the composition contains less than 20% w/w free fatty acids. According to some embodiments, the composition contains less than 20% w/w fatty acid ethyl esters.

According to some embodiments, the composition further comprises a phytocannabinoid. According to some embodiments, the phytocannabinoid is cannabidiol (CBD) and/or tetrahydrocannabinol (THC).

According to some embodiments, the fatty acids of the compositions are in the form of: 10-30%, or 15-25% monoglycerides, 40-80% or 50-70% diglycerides, and 10-30% or 12-20% triglycerides, 1-10%. According to some embodiments, the fatty acids of the compositions further include 1-10% or 2-5% free fatty acids. According to some embodiments, the fatty acids of the compositions further include 1-10% or 2.5-7% fatty acids ethyl esters. According to some embodiments, the triglycerides of the composition include 15-50% or 25-40% EPA and 10-30% or 15-25% DHA.

According to some embodiments, the composition contains at least about 0.0001%, 0.001%, 0.01%, 0.1%, 1% or 10% (w/w) phytocannabinoid. Each possibility is a separate embodiment. According to some embodiments, the composition contains about 0.01%-1%, 0.1%-10% or 1%-15% (w/w) phytocannabinoid. According to some embodiments, the composition contains 10-50 mg phytocannabinoid per unit/dose. Each possibility is a separate embodiment. According to some embodiments, the mono- and di-glycerides containing at least 20% (w/w) omega-3 fatty acyl groups increase the bioavailability of the phytocannabinoid.

According to some embodiments, the composition is suitable for use in increasing omega-3 fatty acid- based endocannabinoids in a subject. According to some embodiments, the composition is suitable for use in increasing mono-, di- and optionally also tri-unsaturated fatty acid-based endocannabinoids in a subject.

According to some embodiments, the composition is suitable for use in treating inflammation, such as but not limited to pancreatic inflammation. According to some embodiments, the inflammation is liver inflammation.

According to some embodiments, the composition is suitable for oral, intraperitoneal (IP), intravenous (IV), or subcutaneous (SC) administration. Each possibility is a separate embodiment.

On this basis, the pharmaceutical composition recited herein may include, albeit not exclusively, solutions of the active ingredients in association with one or more pharmaceutically acceptable vehicles or diluents and may be contained in buffer solutions with a suitable pH and iso-osmotic with physiological fluids.

According to some embodiments, the composition further contains pharmaceutically acceptable concentrations of salt, buffering agents, preservatives and various compatible carriers. Each possibility is a separate embodiment.

According to some embodiments, the composition may be in the form of an emulsion, a suspension, liposomes, encapsulated or confined in hydrogels.

According to some embodiments, there is provided a method for treating inflammation in a subject in need thereof, the method comprising administering to the subject a composition comprising mono- and di-glycerides containing at least 20% (w/w) omega-3 fatty acyl groups, as essentially described herein, thereby reducing the level of inflammation in the subject.

According to some embodiments, reducing the level of inflammation comprises reducing the level of blood serum hydrolases. According to some embodiments, the blood serum hydrolases comprise lipase and/or amylase.

According to some embodiments, the administering comprises oral, intraperitoneal (IP), intravenous (IV), or subcutaneous (SC) administration. Each possibility is a separate embodiment.

According to some embodiments, there is provided a method for use in increasing omega-3 fatty acid- based endocannabinoids in a subject, the method comprising administering to the subject a composition comprising mono- and di-glycerides containing at least 20% (w/w) omega-3 fatty acyl groups, as essentially described herein, thereby increasing the level of omega-3 fatty acid- based endocannabinoids in the subject.

According to some embodiments, there is provided a method for increasing bioavailability of a phytocannabinoid, the method comprising administering to the subject a composition comprising mono- and di-glycerides containing at least 20% (w/w) omega-3 fatty acyl groups, as essentially described herein, thereby increasing the level of omega-3 fatty acid- based endocannabinoids in the subject.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### Example 1 - Administration of Mono- and Di-Glycerides With Omega-3 Fatty Acyl Groups Increases Levels of Endocannabinoids

Eight mice were used in this test. Two mice served as a Control and the remaining 6 mice were each injected IP with 400microl of composition A group (set forth below). Three mice were sacrificed after 1 hour and the remaining three mice were sacrificed 4 hours after the injection. Endocannabinoid concentrations were analyzed in blood serum as well as in the brain of all mice. Composition A (based on Gas Chromatography area ratios) is as set forth:

| | |
|---|---|
| Monoglycerides: | 16% |
| Diglycerides: | 60% |
| Triglycerides: | 14.5% |
| EPA: | 31.5% |
| DHA: | 21% |
| Free Fatty Acid Value (FFA): | 4% |
| Fatty Acids Ethyl Esters (FAMEs): | 5.5% |

Tables 1 and 2 below show the concentrations of the main endocannabinoids which dramatically increased in both blood serum as well as in the brains of the mice 1 and 4 hours after administering IP the herein disclosed composition containing omega-3 fatty acids in the form of mono- and di-glycerides, as compared to control where saline was injected instead.

TABLE 1

Concentrations of endocannabinoids in blood serum of mice after IP administration of omega-3 fatty acids in the form of mono- and di-glycerides as compared to saline injected controls

| Endocannabinoid | Average concentration in blood in control mice (ng/ml) | Average concentration in blood 1 hour after administration of omega-3 composition (ng/ml) | Average concentration in blood 4 hours after administration of omega-3 composition (ng/ml) |
|---|---|---|---|
| EPEA | 0 | 0.4 | 0.3 |
| EP-Gly | 0 | 1.4 | 0.6 |
| DHEA | 0.1 | 1 | 0.9 |
| 2-EPG | 5.5 | 40500 | 10100 |
| 2-LnG | 2 | 600 | 150 |
| AEA | 0.2 | 0.25 | 0.2 |
| DH-Gly | 0 | 3.2 | 2.1 |
| 2-DHG | 12 | 36000 | 13000 |
| 2-AG | 6.2 | 2200 | 600 |
| OEA | 0.4 | 0.4 | 0.85 |
| O-Gly | 0.2 | 0.5 | 0.4 |
| DH-Leu | 0 | 0.9 | 3.2 |
| 2-PG | 1700 | 5100 | 4100 |
| 2-OG | 300 | 10500 | 5300 |

Abbreviations: EPEA: N-Eicosapentaenoyl ethanolamine, EP-Gly: N-Eicosapentaenoyl glycine, DHEA: N-Docosahexaenoyl ethanolamine, 2-EPG: 2-Eicosapentaenoyl glycerol, 2-LnG: 2-Linoleoyl glycerol, AEA: N-Arachidonoyl ethanolamine, DH-Gly: N-Docosahexaenoyl glycine, 2-DHG: 2-Docosahexaenoyl glycerol, 2-AG: 2-arachidonoyl glycerol, OEA: N-oleoyl ethanolamine, O-Gly: N-Oleoy glycine, DH-Leu: N-Docosahexaenoyl leucine, 2-PG: 2-Plamitoleoyl glycerol, 2-OG: 2-Oleoy glycerol.

TABLE 2

Concentrations of endocannabinoids in brains of mice after IP administration of omega-3 fatty acids in the form of mono- and di-glycerides as compared to saline injected controls.

| Endocannabinoid | Average concentration in brain of Control mice (ng/ml) | Average concentration in brain 1 hour after administration of omega-3 composition (ng/ml) | Average concentration in brain 4 hours after administration of omega-3 composition (ng/ml |
|---|---|---|---|
| EPEA | 0 | 0 | 0.2 |
| EP-Gly | 0 | 2.1 | 2.1 |
| DHEA | 40.2 | 50.2 | 40.8 |
| 2-EPG | 4.5 | 2450 | 52.5 |
| 2-LnG | 0 | 25.5 | 1.5 |
| AEA | 57 | 60.2 | 51.5 |
| DH-Gly | 6.2 | 5.9 | 5.2 |
| 2-DHG | 440 | 2950 | 640 |
| 2-AG | 850 | 1221 | 1150 |
| OEA | 124 | 132 | 102 |
| O-Gly | 4.2 | 4.7 | 3.8 |
| DH-Leu | 0 | 0 | 0 |
| 2-PG | 7102 | 9200 | 8350 |
| 2-OG | 4420 | 7430 | 5620 |

Abbreviations: See Table 1

### Example 2 - Administration of Mono- and Di-glycerides With Omega-3 Fatty Acyl Groups Reduces the Level of Blood Serum Hydrolases

Eight mice were used in this test:
1) 1 mouse was injected with 400 micol saline to serve as a Control (saline).
2) 2 mice were injected with cerulein in order to induce pancreatic inflammation and serve as control 2 (CER).
3) 1 mouse was injected with cerulein in order to induce pancreatic inflammation and then injected subcutaneously with 400 microliters with a composition containing mono- and di-glycerides with omega-3 fatty acyl groups as disclosed in example 1 (A-SC).
4) 2 mice were injected with cerulein in order to induce pancreatic inflammation and then injected IP with 400 microliters with a composition containing mono- and di-glycerides with omega-3 fatty acyl groups, as disclosed in example 1 (A-IP).

All mice were sacrificed after 24h for analysis of lipase and amylase activity in their blood serum, which activation served as an indication for pancreatic inflammation, in addition to determining triglycerides and cholesterol concentrations.

The results are shown in table 3 below.

As seen from the table, in mice administered with cerulin only (CER) a significant increase in levels of amylase and lipase was observed in the blood, as expected due to the development of pancreatitis. However, injection of the herein disclosed compositions containing mono- and di-glycerides with omega-3 fatty acyl groups, resulted in a significant reduction in amylase and lipase blood levels, thus indicating amelioration of the pancreatitis.

TABLE 3

blood serum amylase, lipase, triglycerides and cholesterol levels in blood serum (BL)

| | | Amylase-BL (micromole/s-L) | Triglycerides-BL (mg/dL) | Lipase-BL (micromole/s-L) | Cholesterol-BL (mg/dL) |
|---|---|---|---|---|---|
| Saline | | 1403 | 189 | 90 | 90 |
| CER | 6628 | 99 | 382 | 76 | |
| CER | 3446 | 126 | 224 | 79 | |
| A-SC | 3164 | 124 | 191 | 86 | |
| A-IP | 2746 | 283 | 61 | 126 | |
| A-IP | 1469 | 80 | 67 | 103 | |

### Example 3 - Administration of Mono- and Di-glycerides With Omega-3 Fatty Acyl Groups Increase the Bioavailability of Phytocannabinoids

The use of Enzymega for increasing the bioavailability of CBD and THC was tested on rats using fish oil triglycerides containing similar amount of EPA and DHA as a control, as set forth in Table 4 below.

TABLE 4

Experimental outline

| Group | Administration route | Dose of CBD/THC (mg/kg) per rat | Matrix | No. of animals |
|---|---|---|---|---|
| 1 | Oral | 10/8 | Fish oil (Triglycerides) | 6 |
| 2 | Oral | 10/8 | Enzymega | 6 |

Blood draws: Time points: BL: 15 min, 30 min, 1 hr, 2 hr, 3 hr, 4 hr, 6 hr, 8 hr, 12 hr, 24 hr (total of 10 time points).

Dose/Route: 60 mg for a mixture 1:1 by weight of CBD (99% CBD - product of Tikun Olam) and THC (80% purity) per KG of rat by oral administration.

Dose frequency: Single dose of 0.45 ml for each rat (containing 10 mg CBD and 8 mg THC for each rat).

Blood processing: Blood will be collected in VACUTEST tube Sterile R interior, with K⅔ EDTA. Samples are then subjected to centrifugation at room temperature at 4000 rpm for 10 minutes. After Centrifugation, the upper plasma is collected ad stored at -70° C. until further use.

Bioanalytical evaluation: CBD and THC by LCMS/MS, analysis were conducted by Analyst Ltd., Kiryat Weizman, Ness Ziona).

Table 5 below shows the kinetic profile of CBD and THC in blood serum of rats orally injected with similar concentrations of CBD and THC dissolved in Enzymega or in fish oil triglycerides as a control.

TABLE 5

The THC and CBD kinetic profile in blood serum of rats after injection of 60 mg for a mixture 1:1 by weight of CBD (99% CBD - product of Tikun Olam) and THC (80% purity) per KG of rats by oral administration.

| | THC (ng/ml) | THC (ng/ml) | CBD (ng/ml) | CBD (ng/ml) |
|---|---|---|---|---|
| Time (hours) | Fish oil | Enzymega | Fish oil | Enzymega |
| 0 | 0.00 | 0.00 | 0.00 | 0.00 |
| 0.5 | 179.52 | 162.50 | 87.02 | 80.27 |
| 1 | 221.63 | 395.49 | 74.75 | 161.52 |
| 2 | 362.84 | 423.60 | 101.00 | 121.48 |
| 4 | 506.69 | 789.36 | 147.96 | 221.97 |
| 6 | 829.34 | 600.46 | 225.75 | 137.05 |
| 12 | 415.96 | 687.96 | 94.11 | 170.22 |

The results presented in Table 4 show that use of Enzymega instead of fish oil triglycerides both including equivalent amount of omega-3 fatty acids, yield improved bioavailability of CBD and THC with delayed higher concentrations of both components in blood serum after 12 hours from administration.

### Example 5 - Pharmacokinetic Profile for the Absorption of EPA and DHA

The objective of this pre-clinical study was to assess the pharmacokinetic profile for the absorption of EPA and DHA in the plasma of rats administered with the hereindisclosed composition (hereinafter “Enzymega” or with one of three commercially available products. The fatty acid profile and composition of Enzymega is outlined below:

| | |
|---|---|
| EPA: 32.4% of total fatty acids | Palmitic acid (C16:0): 8.3% |
| DHAs: 24.6% of total fatty acids | Palmitoleic Acid (16:1): 6.4% |
| Myristic acid (C14:0): 4.5% | Oleic acid (18:1): 9.4% |

Others: 14.4 %
The fatty acids are in the form of:
Monoglycerides (MGs): 27.7%
Diglycerides (DGs): 47.96%
Triglycerides (TGs): 6.66%
Fatty acids ethyl esters (FAEEs): 9%
Free fatty acids (FFAs): 8.66%

Methods: Rats were administered Per-Os with the Enzymega, or with one of the three commercial formulations, as outlined in table 6 below.

TABLE 6

Concentration of Active Ingredient

| Test Material | Active Ingredient | Active Ingredient Concentration (%) | Oil Density (g/mL) |
|---|---|---|---|
| Soybean oil (Vehicle) | NA | NA | 0.88 |
| Fish Oil A 18/12 (Commercial oil) | Omega 3 | 30 | 0.89 |
| Fish Oil B 18/12 (Commercial oil) | Omega 3 | 30 | 0.98 |
| Ethyl Ester (50%) (Commercial oil) | Omega 3 | 50 | 0.87 |
| Enzymega (Test Item) | Omega 3 | 50 | 0.90 |

NA - Not applicable

Blood was collected from the submandibular vein (0.15-0.25 mL), for DHA, EPA, and higher volume of blood was collected 24 hours post administration before sacrifice, for DHA, EPA and triglycerides (TG) and cholesterol determinations. Three animals from each group were bled at three timepoints: baseline, 1.5 and 8 hours, and the other three animals were bled at 30 min, 4 and 24 hours, as outlined in table 7 below. At all bleeding timepoints, the blood was collected tubes plasma separated. In the last timepoint (24 hours), additional bleeding was collected into tubes with clotting activator gel and the serum was separated. For plasma separation: the tubes centrifuged at 2-8° C. for 10 minutes at 1790 g. For serum separation: the tubes were kept at RT for at least an hour for clotting and subsequently centrifuged at RT for 10 minutes at 1790 g. Serum and plasma were separated and collected into Eppendorf tubes (or equivalent). Plasma was stored at -60° C. to -90° C., 100 mL per sample until being transferred for DHA, EPA bioanalysis, and serum samples (250 mL per sample) were stored at 2-8° C. for Cholesterol and TG analysis.

TABLE 7

Group Allocation

| Group | No. of Animals | Treatment | Dose Level (mg/kg) | Dose Volume (mL/kg) | ROA | Bleeding timepoints |
|---|---|---|---|---|---|---|
| 1 M | ^{#}1,2,3, ^{##}4,5,6 | Soybean oil (Vehicle) | *NA | 1.33 | Oral | ^{#}3 animals: 0, 1.5, 8 hours. ^{##}3 animals: 30 min, 4 and 24 hours. |
| 2 M | ^{#}7,8,9, ^{##}10,11,12 | Fish Oil A 18/12 (Commercial oil) | 350 | 1.32 | | |
| 3 M | ^{#}13,14,15, ^{##}16,17,18 | Fish Oil B 18/12 (Commercial oil) | | 1.19 | | |
| 4 M | ^{#}19,20,21, ^{##}22,23,24 | Ethyl Esters (Commercial oil) | | 0.80 | | |
| 5 M | ^{#}25,26,27, ^{##}28,29,30 | Enzymega (Test Item) | | 0.78 | | |

ROA - Route of Administration; M - Male; NA - Not applicable (*there was no active ingredient in the vehicle)

Pharmacokinetic parameters were calculated using the computer program PK Solutions 2.0 (Summit Research Services, USA).

Maximum observed plasma concentrations (Cₘₐₓ) and their times of occurrence (Tₘₐₓ) were the observed values. Areas under plasma concentration-time curves up to the last quantifiable concentration (AUC₀₋ₜ) were calculated using the linear trapezoidal rule. In the calculation of AUC₀₋ₜ values, it was assumed that the pre-dose (0 hours) plasma concentrations were zero. AUC_{inf} was calculated using by combining AUC₀₋ₜ with an extrapolated value (Cn/λ_{Z}). Data permitting, the terminal elimination rate constant (λ_{Z}) was estimated by fitting a linear regression of log concentration against time and t_{½} was calculated as ln2/λ_{Z}. For estimation of λ_{Z} to be accepted as reliable, the following criteria were set:
1. The terminal data points were apparently randomly distributed about a single straight line (on visual inspection).
2. A minimum of three data points were available for the regression.
3. The regression coefficient was ≥0.85.
4. The interval including the data points chosen for the regression was at least two-fold greater than the half-life itself.

Results: No morbidity was observed in any of the groups during the in-life period. Body weight (BW) and no differences in clinical signs monitored was observed

Advantageously, as seen from table 8, the level of triglycerides mean value in the Enzymega treated group (5 M, 129 ±19.4 mg/dL) was higher than all other tested groups, and significantly higher than the Soybean vehicle group (1 M, 82±18 mg/dL). In addition, it was above the maximum value of triglycerides level (86 mg/dL, according to historical data of AML).

TABLE 8

Triglycerides levels
Triglycerides (mg/dL)

| Group | Raw data | AVG | SD | SEM | T-test vs. Enzymega |
|---|---|---|---|---|---|
| Soybean oil (Vehicle)(1 M) | 87 | 82 | 18.0 | 10.4 | p<0.05 |
| | 97 | | | | |
| | 62 | | | | |
| Fish Oil A 18/12 (Commercial oil) (2 M) | 77 | 94* | 15.6 | 9.0 | ns |
| | 96 | | | | |
| | 108 | | | | |
| Fish Oil B 18/12 (Commercial oil) (3 M) | 107 | 95* | 17.2 | 9.9 | ns |
| | 75 | | | | |
| | 102 | | | | |
| Ethyl Ester (Commercial oil) (4 M) | 74 | 89* | 18.9 | 10.9 | ns |
| | 110 | | | | |
| | 82 | | | | |
| Enzymega (Test Item) (5 M) | 134 | 129* | 19.4 | 11.2 | NA |
| | 108 | | | | |
| | 146 | | | | |
| Historical data | Min | 21 | | | |
| | Max | 86 | | | |

M - male; AVG - Average; SD - Standard deviation; SEM - Standard error of the mean; ns - Non significant; NA - Not Applicable.
*values above the maximum value according to historical data

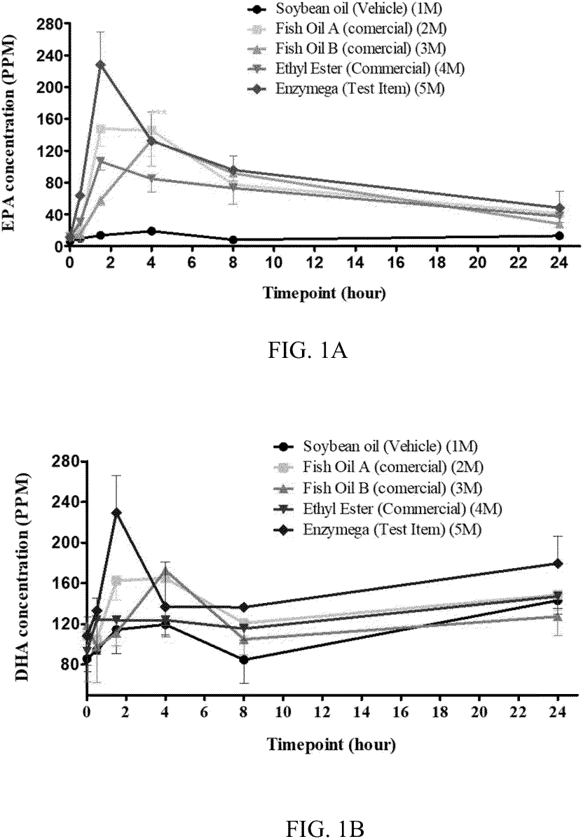

Furthermore, As seen from FIG. 1A and FIG. 1B, a significantly improved absorption profile was observed in rats administered with Enzymega as compared to all other tested formulation. Specifically, absorption (Cₘₐₓ) and the AUC was higher for both EPA and DHA and the absorption time (Tₘₐₓ) was lower for both analytes in rats administered with Enzymega.

### Example 6 - Liver Inflammation

The objective of this study was to assess the effect of the herein disclosed composition on liver inflammation/injury using CCl4 mouse model of liver injury.

In order to evaluate the effect of the herein disclosed composition (hereinafter “ENZYMEGA”) on liver inflammation, mice (N=64) were divided into two main groups a control group which were not administered with CCl4 (N=32) and a CCl4 treated group (N=32) at concentration of 0.008 mg/lg mice in day 1, Day 3 and Day 5. The two main groups were further subdivided into 2 subgroups that were administered with the compositions and as outlined in table 9 below.
1. Negative control: Soybean oil - SBO.
2. Enzymega tested group

TABLE 9

Administration scheme

| | | | | |
|---|---|---|---|---|
| Day 1 | Day 3 | Day 5 | Day 6 | Day 7 |
| CCl4 + composition (150 microliters of SBO and 0.008 mg/lg mice, or 150 microliters of Enzymega and 0.008 mg/lg mice) | CCl4 + composition (150 microliters of SBO and 0.008 mg/lg mice, or 150 microliters of Enzymega and 0.008 mg/lg mice) | CCl4 + composition (150 microliters of SBO and 0.008 mg/lg mice, or 150 microliters of Enzymega and 0.008 mg/lg mice) | Composition (150 microliters of SBO, or 150 microliters of Enzymega) | Sacrificing |

The mice were monitored for weight and food-water intake and no significant differences were observed between the groups.

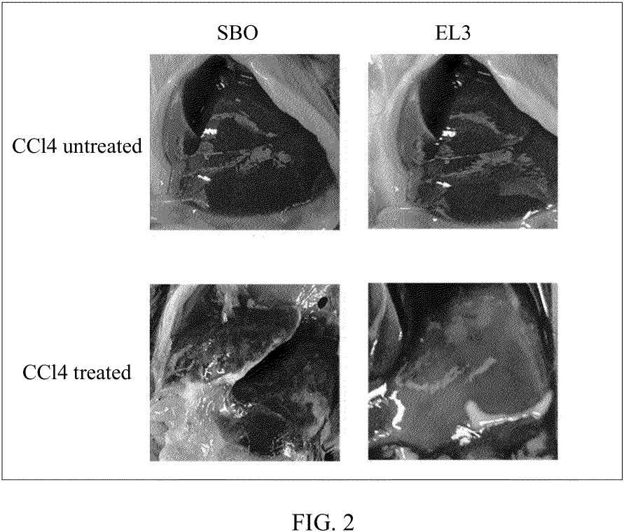

However, as seen from FIG. 2 a significant difference was observed with respect to liver health. As expected, CCl4 caused significant liver inflammation, as compared to CCl4 untreated livers, but the health of the liver was essentially restored in livers treatment with Enzymega.

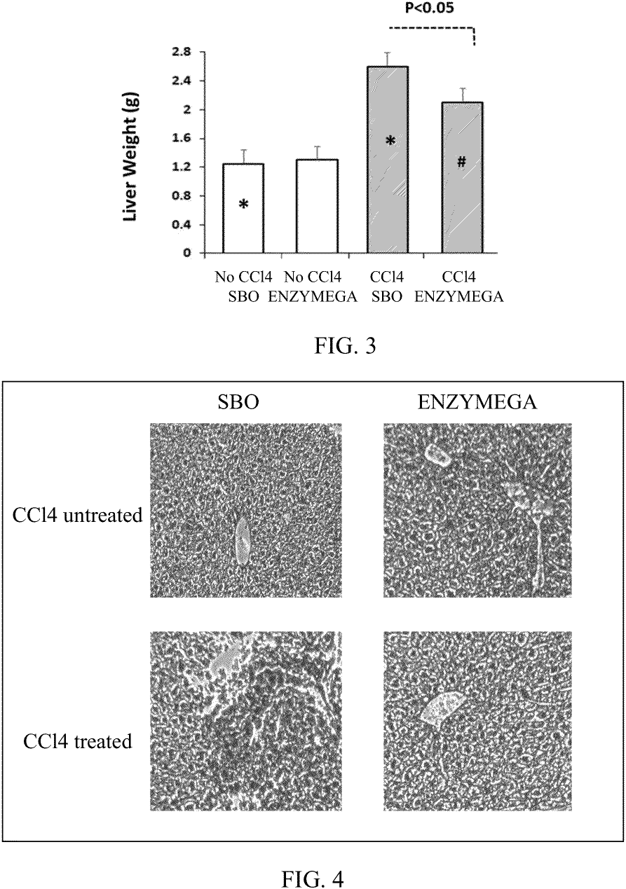

Moreover, while treatment with CCl4 in the presence of soybean oil resulted in a significant increase in live weight, treatment with ENZYMEGA attenuated this increase (by almost 20%), while having no impact on livers which were not induced for inflammation (FIG. 3).

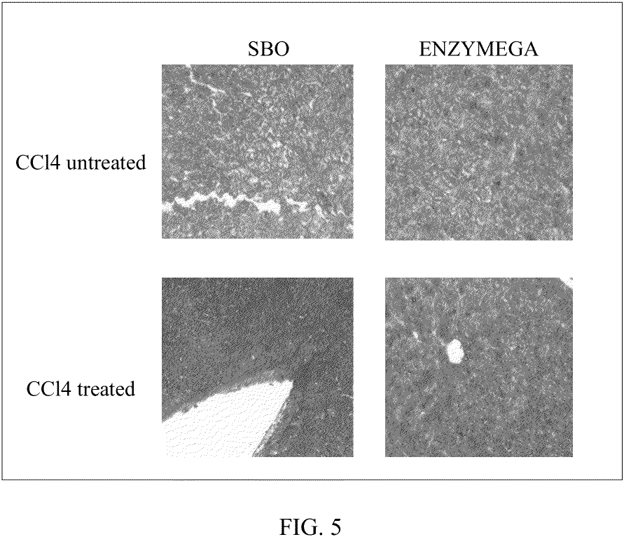

Hematoxylin and eosin (H&E) stains of liver tissue showed swelled centrilobular hepatocytes and large necrotic areas of high infiltrating inflammatory cells with steatosis in tissue from mice treated with CCl4 in combination with soybean oil (negative control), while the tissue of mice, which received ENZYMEGA along with the CCl4 treatment, resembled that of CCL4 untreated mice (FIG. 4). Similarly, Sirius Red staining showed increased collagen deposition in perisinusoidal areas in tissue from mice treated with CCl4 in combination with soybean oil (negative control), while the tissue of mice, which received ENZYMEGA along with the CCl4 treatment, resembled that of CCL4 untreated mice (FIG. 5).

Alanine aminotransferase (ALT) and aspartate aminotransferase (AST) are enzymes released from the liver hepatocytes in cases of toxins in the liver, liver disease, or liver damage. ALT is only found in livers while AST is normally found in a variety of tissues including the liver, heart, muscle, kidney, and brain. It is released into the serum when any one of these tissues is damaged. For example, the AST level in serum is elevated in heart attacks or as a result of muscle injury.

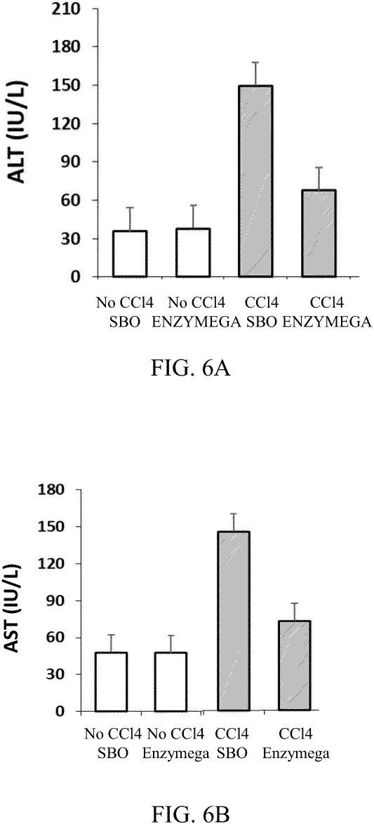

Importantly, as seen from FIG. 6A and FIG. 6B, both ALT and AST levels increased significantly in the blood of mice treated with CCl4 in combination with soybean oil (negative control). However, the increase in the levels of both enzymes were advantageously significantly attenuated in mice that received ENZYMEGA along with the CCl4 treatment.

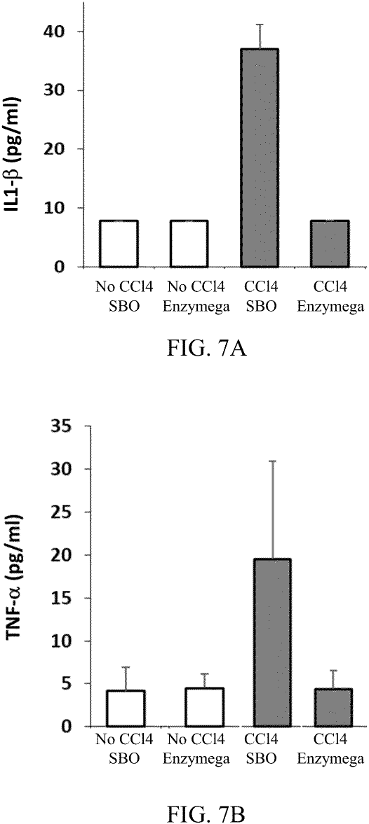

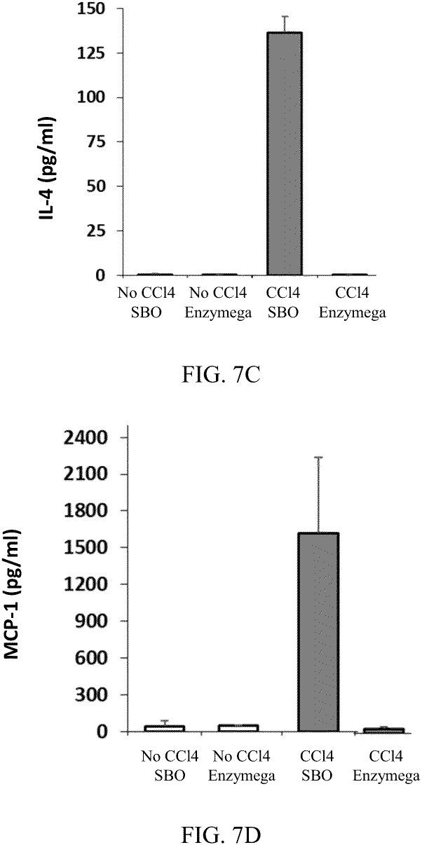

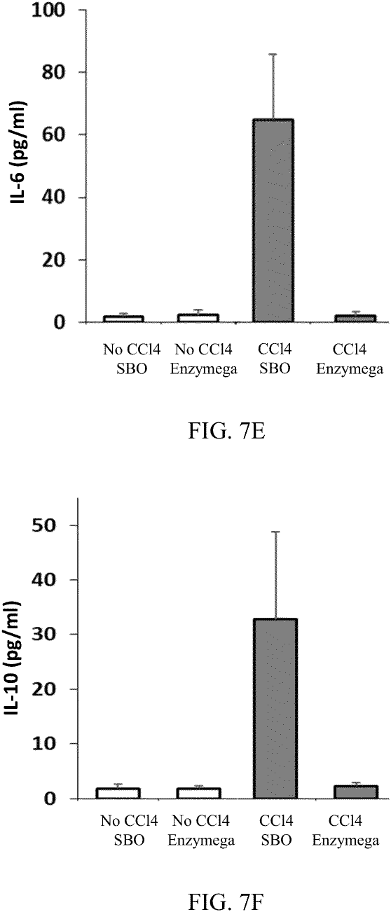

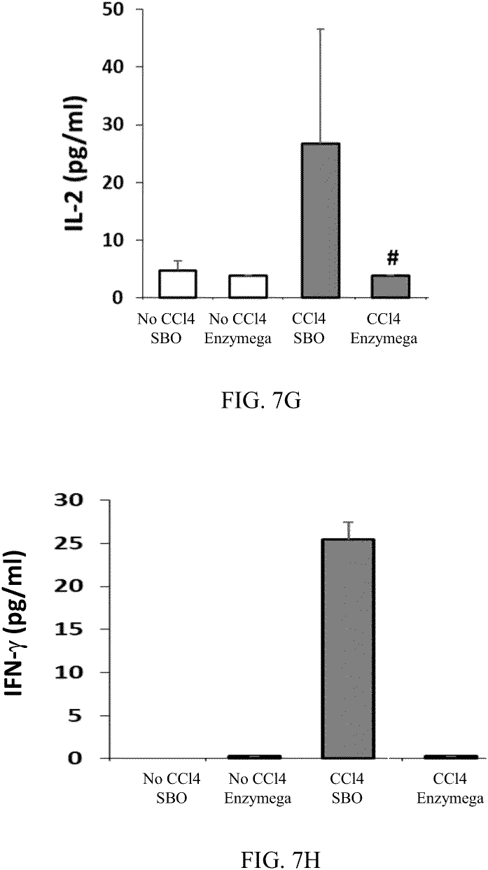

A similar observation was found when the levels of inflammatory cytokines was evaluated (IL1-β - FIG. 7A; TNF-α - FIG. 7B; IL-4 - FIG. 7C; MCP-1 - FIG. 7D; IL-6 - FIG. 7E; IL-10 - FIG. 7F, IL-2 - FIG. 7G, and INF-γ - FIG. 7H).

In particular, it is noted that Enzymega completely restores (reduces) the levels of INF-γ, (FIG. 7H), the expression of which was increased as a result of the liver inflammation (as induced by the CCl4 injection). IFNγ expression is associated with a number of autoinflammatory and autoimmune diseases and is known for its immunostimulatory and immunomodulatory effects.

These result clearly indicates the surprising ability of the herein disclosed composition to inhibit INF-γ expression and to alleviate liver inflammation.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

What we claim is:

1-58. (canceled)

59. A method for increasing the levels of omega-3 fatty acids and omega-3 fatty acid-based endocannabinoids in a subject in need thereof, the method comprising administering to the subject a composition comprising mono- and di-glycerides containing at least 20% w/w of the total product weight of omega-3 fatty acyl groups, thereby increasing the level of the omega-3 fatty acids and the omega-3 fatty acid-based endocannabinoids.

60. The method of claim 59, wherein the composition comprising mono- and di-glycerides contains at least 60% (w/w) omega-3 fatty acyl groups.

61. The method of claim 59, wherein the omega-3 fatty acyl groups comprise eicosapentaenoic acid (EPA) fatty acyl groups, docosahexaenoic acid (DHA) fatty acyl groups or both.

62. The method of claim 59, wherein the composition comprises mono- and di-glycerides containing one or more fatty acyl groups selected from oleic, palmitic, myristic, stearic, arachidonic, palmitoleic, linoleic and linolenic acyl groups.

63. The method of claim 59, wherein the administration of the composition induces an increase of the levels of 2-monoglyceride and/or fatty acid ethanol amides of omega-3 fatty acyl groups such as EPA and DHA, in the subject.

64. The method of claim 63, wherein the administration of the composition induces an increase of one or more biological effects that are mediated primarily by CB1 cannabinoid receptors in the central nervous system, and/or CB2 cannabinoid receptors in the periphery.

65. The method of claim 59, wherein the administration of the composition reduces or prevents one or more undesired biological effect caused by arachidonic acid-based endocannabinoids.

66. The method of claims 59, wherein the composition further comprises tri-glycerides containing at least one omega-3 fatty acyl group, wherein the tri-glycerides comprise at least one omega-3 fatty acyl group.

67. The method of claim 59, wherein the composition comprises less than 20% w/w free fatty acids and/or wherein the composition comprises less than 20% w/w fatty acid ethyl esters.

68. The method of claim 59, wherein the ratio of mono-glycerides and di-glycerides in the composition is in the range of about 1.5:1 - 1:6.

69. A method for treating inflammation in a subject in need thereof, the method comprising administering to the subject a composition comprising mono- and di-glycerides containing at least 20% (w/w) omega-3 fatty acyl groups, thereby reducing the level of inflammation in the subject, wherein reducing the level of inflammation comprises reducing the level of blood serum hydrolases, and/or reducing the level of one or more aminotransferases and/or reducing the level of one or more cytokines.

70. The method of claim 69, wherein the composition comprising mono- and di-glycerides contains at least 60% (w/w) omega-3 fatty acyl groups.

71. The method of claim 69, wherein the composition comprises less than 20% w/w free fatty acids and/or wherein the composition comprises less than 20% w/w fatty acid ethyl esters.

72. A composition comprising mono- and di-glycerides containing at least 20% (w/w) omega-3 fatty acyl groups, wherein the composition comprises at least 10% (w/w) mono-glycerides containing at least 20% (w/w) omega-3 fatty acyl groups; or at least 10% (w/w) di-glycerides containing at least 20% (w/w) omega-3 fatty acyl groups; or wherein the ratio of mono-glycerides and di-glycerides in the composition is in the range of about 1.5:1 -1:6.

73. The composition of claim 72, wherein the mono- and/or di-glycerides comprises at least 50% (ww) omega-3 fatty acyl groups.

74. The composition of claim 72, comprising less than 20% (w/w) free fatty acids and/or less than 20% (w/w) fatty acids ethyl esters.

75. The composition of claim 72, for use in increasing omega-3 fatty acid- based endocannabinoids in a subject and/or in increasing mono-, di- and/or tri-unsaturated fatty acid-based endocannabinoids in a subject and/or in treating inflammation, wherein the inflammation is pancreatic inflammation and/or liver inflammation.

76. The composition of claim 72, wherein the composition comprises at least 10% (w/w) di-glycerides and/or wherein the ratio of mono-glycerides and di-glycerides in the composition is in the range of about 1.5:1 - 1:6.

77. The composition of claim 72, comprising less than 20% w/w free fatty acids.

78. The composition of claim 72, further comprising one or more phytocannabinoid.
